(19) United States
(12) Patent Application Publication
(10) Pub. No.: US 2012/0191171 A1
(43) Pub. Date: Jul. 26, 2012

(54) SUPPORT DEVICE FOR A TUBULAR STRUCTURE

(75) Inventors: Zoran Milijasevic, Bayview (AU); Richard J. Parkinson, Darlinghurst (AU); Johnathon Choi, Carlingford (AU)

(73) Assignee: Murray Vascular PTY limited

(21) Appl. No.: 13/383,206

(22) PCT Filed: Jul. 8, 2010
(86) PCT No.: PCT/AU2010/000873

§ 371 Date: Apr. 13, 2012

(30) Foreign Application Priority Data
Jul. 9, 2009 (AU) 82009903232

(51) Int. Cl. A61F 2/82; A61F 2/01

(52) U.S. Cl. 623/1.11; 623/1.15; 606/200

(57) ABSTRACT

A blood vessel support device (10) includes a body member (12), the body member (12) comprising at least one elongate element (14) of a settable material. The at least one elongate element (14) is configured to change from a non-deployed configuration to be received in an introducer for delivery to a site within a blood vessel to a deployed, coiled, tubular configuration at the site within the blood vessel.

### CROSS-REFERENCE TO RELATED APPLICATIONS

The present application claims priority from Australian Provisional Patent Application No. 2009903232 dated 9 Jul. 2009, the contents of which are incorporated herein by reference in their entirety.

### FIELD

This disclosure relates, generally, to a support device and, more particularly, to a support device for use within a tubular structure such as a blood vessel in a human or animal body. The support device can also be used as a stent within a blood vessel.

### BACKGROUND

In tubular structures weaknesses sometimes exist which require support. These weaknesses could arise for any number of reasons. In medical applications, in tubular structures such as blood vessels, the weakening of a wall of the blood vessel may result from an aneurysm occurring in the blood vessel.

In addition, narrowing of the tubular structure can occur due to a number of factors. Once again, in a medical application, the narrowing of a blood vessel can occur due to build up of plaque in the blood vessel. A support device can be used to inhibit such narrowing of the tubular structure or to inhibit re-occurrence of the narrowing of the tubular structure.

In some applications, the tubular structure is of small cross section and the placement of a support device in such a tubular structure can be difficult. Still further, in some applications, it is desirable to gain access to a space between an outer surface of the support device and an inner wall of the tubular structure to place items between the support device and the tubular structure, the support device being used to hold items in place, at least initially.

### SUMMARY

In a first aspect, there is provided a blood vessel support device which includes a body member, the body member comprising at least one elongate element of a settable material, the at least one elongate element being configured to change from a non-deployed configuration to be carried by an introducer for delivery to a site within a blood vessel to a deployed, coiled, tubular configuration at the site within the blood vessel.

By “coiled” is meant that the elongate element adopts a substantially helical configuration. The turns of the coils need not be circular but may be elliptical, substantially polygonal, or the like. Further, by “deployed” is meant that the elongate element of the device coils to conform to the blood vessel in which it is received, that it coils to conform the blood vessel to the size of the elongate element in its fully deployed configuration or a combination of the two. Depending on the application of the device, the device, when in its deployed configuration, may buttress the vessel in which the device is deployed. In its non-deployed configuration, the elongate element may be substantially unwound or, instead, may be contained in a tightly wound, constricted coiled configuration. The tightly wound, constricted coiled configuration of the elongate element may have a major transverse dimension which is substantially smaller than the major transverse dimension of the elongate element in its deployed configuration.

The, or each, elongate element may be in the form of a wire capable of adopting a coiled configuration. The, or each, wire may be of a settable metal material, such as, but not limited to, a nickel/titanium alloy, a settable steel material, or the like, a settable plastics material, or the like. The elongate element may be hollow to define a passage in which the introducer is received to retain the elongate element in its non-deployed configuration.

When the body member is in its deployed configuration, adjacent turns of the coil may be spaced from each other or they may abut. Further, in other embodiments, at certain places along the length of the body member, in its deployed configuration, adjacent turns of the coil may abut while at other places along the length of the body member, adjacent turns of the coil may be spaced from each other.

The elongate element may have any suitable cross-section from circular to polygonal. It may also be of strip-like, or ribbon, form.

The at least one elongate element may be shaped so that, when the at least one elongate element is in its deployed configuration, at least one opening is formed between adjacent turns of a coil formed by the deployment of the body member, the at least one opening enabling access to be gained to the site through the body member. Instead, the body member may be of a material which permits prising or parting of turns of the coil at a desired position, using a catheter or some other special tool, to enable access to be gained to the site.

The body member may comprise a plurality of interdigitated elongate elements. In use, the elongate elements may be introduced to the site simultaneously or individually. In the latter case, the elongate elements may be introduced at different times. This is particularly useful where restenosis occurs after deployment of an initial elongate element.

The body member may form at least two parts when in its deployed configuration, a primary part and at least one secondary part, the parts being axially spaced and interconnected by a bridging portion. A diameter of the primary part may differ from a diameter of the at least one secondary part. The support device may include two secondary parts extending from a distal end of the primary part to form a bifurcated body member.

The body member may include an adjustment mechanism attached to the body member for adjusting a diameter of the body member when the body member is in its deployed configuration. Further, the body member may include a manipulating formation for enabling the body member to be manipulated and retrieved from the site. In an embodiment, the manipulating formation may be a hook or similar structure arranged at either the proximal end or distal end, or both, of the body member. The hook may be engaged and the body member withdrawn into the introducer, which may be tubular, and converted to its non-deployed configuration as it is withdrawn into the introducer. In another embodiment, the manipulating formation may be an elongate member extending proximally from the body member of the support device, a proximal end of the elongate member being accessible externally of a patient's body.

Still further, the body member may include an engageable formation to be engaged by an engaging member to assist in arranging the body member at least in its deployed configuration. The engageable formation may be a hooked formation, or the like, and may be arranged at a distal end of the body member to be engaged by the engaging member to remove the body member from the introducer. The engaging member may be a stylet, or the like. It will be appreciated that what has been described above relates to relative movement between the body member and the introducer. Thus, the body member may be “withdrawn” into the introducer by urging the introducer distally with respect to the body member and, conversely, the body member may be “removed from” the introducer by displacing the introducer proximally relative to the body member.

To ensure that the coils of the elongate member are transversely oriented relative to a longitudinal axis of the introducer, a distal portion of the introducer may include a guide formation. The guide formation may comprise a longitudinally extending slot arranged at a distal end of the introducer through which the elongate element exits the introducer.

In a second aspect, there is provided a system for deploying a support device in a passage of a blood vessel, the system including

an introducer; and

a support device, as described above, carried by the introducer with the body member in its non-deployed configuration.

The system may include an engaging member associated with the introducer for engaging the support device to assist in removing the support device from the introducer to enable the support device to adopt its deployed configuration after ejection from the introducer. In the case of a tubular introducer, the engaging member may be receivable in the lumen of the introducer.

In one embodiment, the introducer may be in the form of a delivery catheter, withdrawal of the catheter from a site after delivery of the support device to the site in the passage of the vessel assisting in causing the body member to adopt its deployed configuration.

In another embodiment, the introducer may be a wire about which the support device, in its non-deployed configuration, is mounted in a tightly wound configuration, the support device being released from its non-deployed configuration to expand radially and, optionally axially, into its deployed configuration.

The introducer may be of polygonal cross-section when viewed end-on and, preferably, may have a rectangular, ribbon-like cross-section when viewed end-on.

In a further embodiment, the introducer may be a wire and the support device may be tubular. The support device, in its non-deployed configuration, may be received over the introducer and may be released from a distal end of the introducer by relative movement between the introducer and the support device to enable the support device to adopt its deployed configuration.

A manipulating formation of the support device may protrude from the site to be accessible externally of the patient's body to enable a clinician to access the device, cause the device to adopt its non-deployed configuration and to be withdrawn from the patient's body.

The support device may be a primary stent and the support device may include at least one secondary stent having a body member of a similar configuration to the primary stent, the primary stent and the secondary stent being used together to form a bifurcated stent at the site.

In a third aspect, there is provided a method of deploying a support device in a passage of a blood vessel, the method including

delivering the support device, as described above, to a site in the vessel using an introducer, the body member of the support device being in its non-deployed configuration; and

ejecting the support device from a distal end of the introducer to enable the support device to adopt its deployed configuration.

The method may include withdrawing the introducer from the site in the vessel at the same time as ejecting the support device from the introducer to aid in the body member of the support device adopting its deployed configuration.

The method may include ejecting only a portion of the support device from the introducer so that that portion of the support device adopts its deployed configuration, the portion of the support device ejected being governed by a size of a region of the blood vessel to be supported. In the case of a support device to be used to buttress a blood vessel in the region of an aneurysm, the size of the portion of the support device which is ejected may be governed by the size of a neck of the aneurysm. Instead, the method may include selecting a size of support device suitable for the size of the region of the blood vessel to be supported.

Further, the method may include engaging an end of the body member of the support device to assist in ejecting the body member from the introducer.

The method may include accessing a manipulating formation of the support device protruding from the site, causing the device to adopt its non-deployed configuration and withdrawing the device from a patient's body.

The method may include ejecting a first support device from the introducer at the site and, thereafter, ejecting a second support device from the introducer so that the second support device adopts a desired position relative to the first support device at the site. The method may include connecting the second support device to the first support device to retain the support devices in position relative to each other. More particularly, the method may include connecting the second support device to the first support device by interdigitating at least a portion of the second support device with a portion of the first support device.

In an embodiment, the method may include arranging the second support device relative to the first support device to form a bifurcated support device. Additional support devices may be deployed to form additional branches.

If necessary, an inflating device, such as a balloon, may be used to deploy the support device.

In a fourth aspect, there is provided a blood vessel support device which includes an inflatable body member, the body member being annular when viewed end-on.

A wall of the body member may define at least one opening, sealed about its periphery to inhibit escape of inflating fluid through the opening. Preferably, the body member includes a plurality of such openings.

The body member may comprise a plurality of panels, at least some of which are inflatable independently of one another.

In a fifth aspect, there is provided a system for embolising an aneurysm, the system including

an introducer;

a support device, as described above, carried by the introducer with the body member of the support device being carried by the introducer in its non-deployed configuration; and

an introduction catheter, in which embolising equipment is receivable, the support device being dimensioned to hold a distal end of the introduction catheter captive between a wall of the blood vessel and the support device when the support device is in its deployed configuration.

In a sixth aspect, there is provided a method of embolising an aneurysm, the method including

delivering a support device, as described above, to a site in the vessel using an introducer, the body member of the support device being carried in its non-deployed configuration by the introducer during the delivery process;

introducing an introduction catheter containing embolising equipment to the site; and

ejecting the support device from a distal end of the introducer to enable the support device to adopt its deployed configuration and to hold a distal end of the introduction catheter captive between a wall of the blood vessel and the support device in its deployed configuration.

### BRIEF DESCRIPTION OF DRAWINGS

FIG. 1 shows a perspective view of a first embodiment of a blood vessel support device in its deployed, operative configuration;

FIG. 2 shows a schematic, sectional view of an embodiment of a system for deploying a blood vessel support device, the support device being shown in its non-deployed configuration;

FIG. 3 shows a schematic, sectional view of a diseased blood vessel;

FIG. 4 shows a side view of a second embodiment of a blood vessel support device in a deployed configuration;

FIG. 5 shows a perspective view of a third embodiment of a partially formed blood vessel support device;

FIG. 6 shows a schematic representation of the partially formed support device of FIG. 5 received in the diseased blood vessel of FIG. 3;

FIG. 7 shows a schematic representation of the formation of the remainder of the support device in situ in the blood vessel of FIG. 3;

FIG. 8 shows a schematic representation of a complete support device in situ;

FIG. 9 shows a schematic representation of the blood vessel after restenosis has occurred;

FIG. 10 shows a schematic representation of the blood vessel after treatment of the restenosis using a fourth embodiment of a support device;

FIG. 11 shows a perspective view of a fifth embodiment of a support device;

FIG. 12 shows a perspective view of a sixth embodiment of a support device;

FIG. 13 shows a schematic, perspective view of a seventh embodiment of a support device;

FIGS. 14-16 show stages in deploying an eighth embodiment of a support device using a second embodiment of a system for deploying a support device;

FIG. 17 shows an end view of a ninth embodiment of a support device;

FIG. 18 shows a three dimensional view of the support device of FIG. 17;

FIG. 19 shows a three dimensional view of a tenth embodiment of a support device;

FIGS. 20a-20c show, schematically, perspective views of different embodiments of the deployment of an eleventh embodiment of a blood vessel support device;

FIGS. 21a and 21b show, schematically, perspective views of two versions of a twelfth embodiment of a blood vessel support device;

FIG. 22 shows, schematically, a perspective view of a third embodiment of a system for deploying a support device, with the support device in its deployed configuration;

FIG. 23 shows schematically, a side view of the system of FIG. 22 with the support device in its non-deployed configuration;

FIG. 24 shows, on an enlarged scale, an end view of the system of FIG. 23;

FIG. 25 shows, schematically and on an enlarged scale, a bottom view of a distal end of the system of FIG. 23;

FIG. 26 shows, schematically, a perspective view of a fourth embodiment of a system for deploying a support device, prior to deployment of the support device;

FIG. 27 shows, schematically, a perspective view of the system of FIG. 26 with the support device at least partially deployed;

FIG. 28 shows, schematically, a side view of a fifth embodiment of a system for deploying a support device, prior to deployment of the support device, the system including a thirteenth embodiment of a support device;

FIG. 29 shows, on an enlarged scale, a perspective view of a proximal portion of the system of FIG. 28;

FIG. 30 shows, schematically and on a reduced scale, a side view of the system after deployment of the support device; and

FIGS. 31a-31d show stages of an embodiment of a method for embolising an aneurysm using an embodiment of a system for embolising an aneurysm.

### DETAILED DESCRIPTION OF PREFERRED EMBODIMENTS

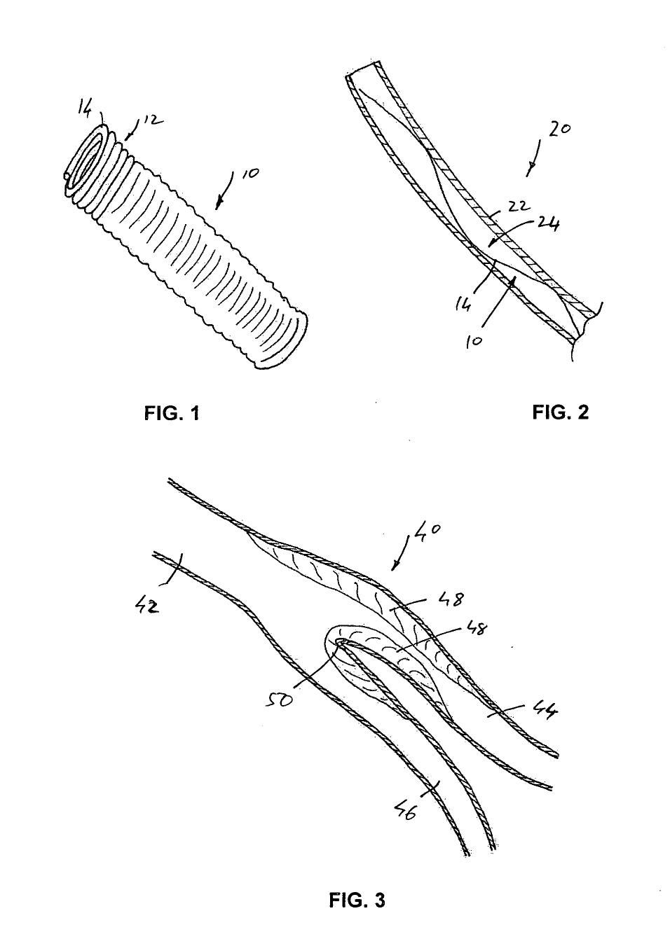

In FIG. 1 of the drawings, reference numeral 10 generally designates an embodiment of a blood vessel support device. In FIG. 1, the support device 10 is shown in an operative, deployed, helically coiled configuration. The support device 10 comprises a body member 12 having at least one elongate element, in the form of a length of wire 14, of a settable material. In a preferred embodiment, the wire 14 is of a settable, metal material but could also be of a suitable plastics material. The wire 14 is formed into a helically coiled configuration in which it is set. The coiled configuration can be unwound into a non-deployed configuration for delivery to a site in a blood vessel to be supported by the support device 10.

The wire 14 is of any suitable cross-section, when viewed end-on, which is capable of being coiled and, depending on the application of the support device 10, the wire 14 can have a polygonal, round, elliptical, or strip-like or ribbon-like cross-section.

Generally, the support device 10 is used in medical applications and is used either as a stent or to support other items in a blood vessel.

As indicated above, the wire 14 is formed from a metal such as, for example, a nickel-titanium alloy (Nitinol). Such an alloy is able to be heat set into a desired configuration. In this case, the wire 14 is formed into the coiled configuration, is set in that configuration and is then unwound to be able to be deployed to a site to be treated in a patient's blood vessel. Instead, as also indicated above, the wire 14 could be of a suitable plastics material such as a suitable polymers. Suitable polymers include polyacetal, polycarbonate, polypropylene, or the like.

The support device 10 is intended generally for use in treating abnormalities such as intracranial aneurysms. The blood vessels within which such aneurysms occur are of the order of 5 mm or less in diameter. Hence, the ratio of a major transverse dimension of the wire 14 used to form the coiled body member 12 and a major transverse dimension of the coil of the body member 12 itself needs to be selected to facilitate coiling of the wire 14 while still performing a support function in the blood vessel. The major transverse dimension of the wire 14 will be dependent on the cross-sectional shape of the wire and is, generally, the thickness of the wire 14. In the case of a wire of circular cross-section, the major transverse dimension is the diameter of the wire 14, for a wire 14 of elliptical cross-section, the major transverse dimension is a major axis of the wire 14, for a wire 14 of square or rectangular cross-section, the major transverse dimension is a diagonal of the wire 14, etc. Similarly, the major transverse dimension of the coiled body member 12 is the outer diameter of the coiled body member 12 where the coiled body member 12 is circular when viewed end-on, a major axis of an elliptical body member 12 when viewed end-on, a diagonal of a substantially square or rectangular body member 12 when viewed end-on, etc.

Broadly a stiffer ratio is selected where the coiled body member 12 is to conform the blood vessel to the shape of the coiled body member 12 and, conversely, a softer ratio is selected where the coiled body member 12 is to conform to the shape of the blood vessel. In the former case, a ratio of 40 or less is appropriate and, in the latter case, a ratio of more than 40 is appropriate.

If the ratio is selected so that the coiled body member 12 is too “soft”, coiling of the body member 12 may not occur when the body member 12 is ejected from an introducer to adopt its deployed configuration. Further, if the body member 12 is too “soft”, the deployed body member 12 may not adequately support or buttress the blood vessel in which it has been deployed.

Conversely, if the coiled body member 12 is too “stiff”, it may not be possible to deploy the body member 12 and/or, when the body member 12 is deployed, it may not coil adequately or it may damage the blood vessel in which it has been deployed.

Thus, a “medium” ratio of coil major transverse dimension to wire major transverse dimension is preferred. A “soft” ratio is of the order of 40 or above, a “hard” or “stiff” ratio will be of the order of 15 or less with a “medium” ratio falling between the two. Examples of ratios are set out in the table below:

| d | D | Ratio-D/d | Stiffness |
|---|---|---|---|
| 0.3 | 3.9 | 13.0 | Hard |
| 0.19 | 3.9 | 20.5 | Med |
| 0.24 | 3.9 | 16.3 | Med |
| 0.12 | 5 | 41.7 | Soft |

where:
d = wire major transverse dimension or thickness of wire;
D = coil major transverse dimension.

Preferred ranges of ratio thus may be between about 15 to 20; 20 to 25; 25 to 30; 30 to 35 and 35 40 depending on the application of the support device 10.

The body member 12 can be formed with adjacent turns of the coil either in abutment, as shown in FIG. 1 of the drawings, or with the coils spaced from each other as shown, for example, in the embodiments illustrated in FIGS. 6-9 of the drawings. In the case where adjacent coils of the body member 12 are spaced from each other, it will be appreciated that openings are provided between adjacent turns so that access can be gained to a wall of the blood vessel or a cavity in the blood vessel arranged outwardly of the support device 10. This enables items such as, for example, embolic coils to be placed through the support device 10 to assist in coagulating blood contained, for example, in an intracranial aneurysm.

An embodiment of a support device 10 used in such a procedure, i.e. for delivering of embolic coils to an intracranial aneurysm is shown in FIG. 12 of the drawings. The spacing between adjacent turns of the coiled body member 12 may be sufficient for placement of the embolic coils or other items. In addition, at least some of the turns 14.1 and 14.2 of the coiled body member 12 which are adjacent each other are shaped to form an opening 26 through which the items can be placed between the support device 10 and a wall of the blood vessel. Instead of a pre-formed opening 26, the opening may be formed while the support device 10 is in situ by deforming adjacent turns of the coiled body member 12 or by prising apart the adjacent turns of the body member 12.

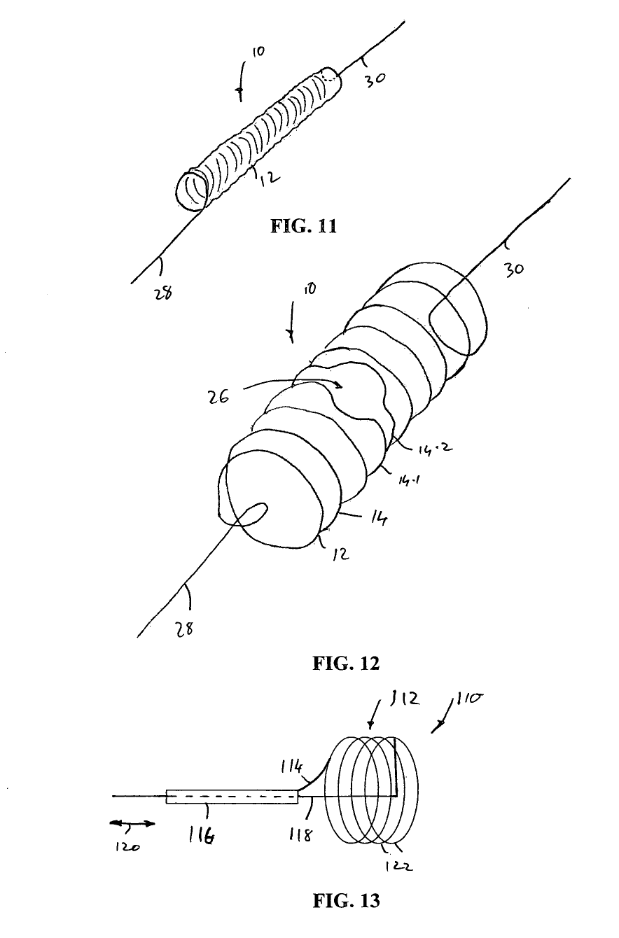

Further, as shown, the support device 10, in this embodiment, as well as in the embodiment shown in FIG. 11 of the drawings, has a manipulating formation in the form of uncoiled trailing and leading members 28 and 30 extending proximally and distally of the body member 12, respectively. At least the member 28, which functions as a retrieval wire, is sufficiently long to protrude from the patient's body to be gripped by a clinician to retrieve the support device 10 from the blood vessel and to leave the items such as the embolic coils in place in the blood vessel.

The member 30 is used as a guide wire and protrudes distally from a delivery catheter (not shown). The member 30 can, optionally, be covered with a covering member such as a tightly wound coil (not shown) to inhibit damage to the blood vessel by the member 30 but having the flexibility similar to a conventional guide wire.

Referring to FIG. 2, an embodiment of a system for deploying a support device is illustrated and is designated generally by the reference numeral 20. The system 20 includes an introducer in the form of a delivery catheter 22 defining a lumen 24. For delivery to the site in the patient's blood vessel, the support device 10 is inserted into the lumen 24 of the delivery catheter 22 by being stretched or unwound into a non-deployed configuration as shown in FIG. 2 of the drawings. The member 28 protrudes proximally from the lumen 24 of the catheter 22.

Another use for the support device 10 is as a stent. FIG. 3 shows a bifurcated blood vessel 40. The blood vessel 40 has a primary branch 42 with two secondary branches 44, 46 leading off the primary branch 42. Narrowing of the blood vessel 40 can occur due to the build up of plaque 48 on the walls of the blood vessel 40. A problem arises where the plaque 48 occurs at the junction 50 between the secondary blood vessels 44 and 46 and the primary blood vessel 42. This problem necessitates the use of a bifurcated stent to achieve stenting at the junction 50 between the secondary blood vessels 44 and 46 and the primary blood vessel 42.

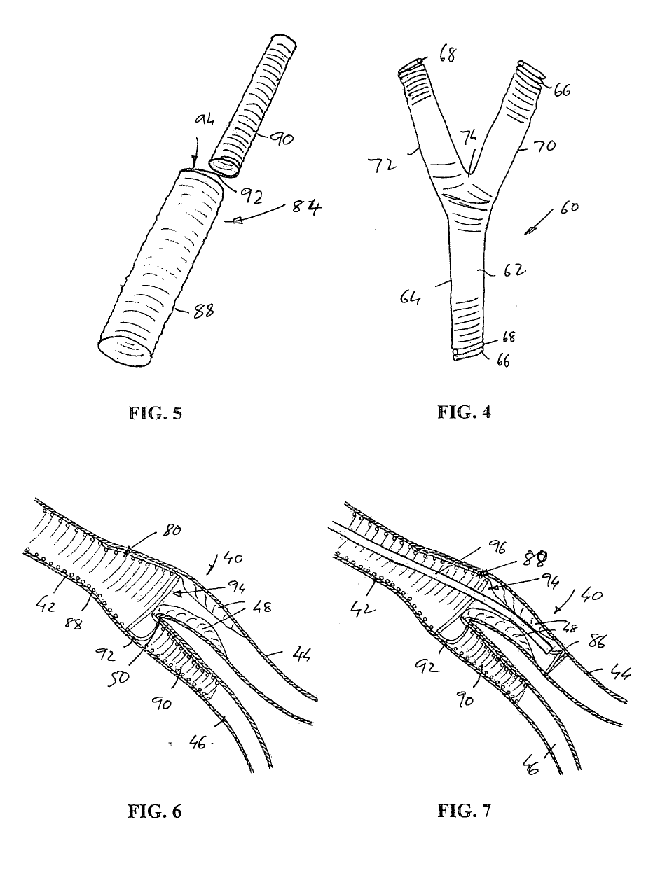

An embodiment of a bifurcated stent is shown in FIG. 4 of the drawings and is designated generally by the reference numeral 60. This stent 60 comprises a primary arm 62 formed with a double, substantially helically coiled configuration. In other words, a body member 64 constituting the stent 60 has two wires formed into coils 66 and 68 which are interdigitated with each other to form the primary arm 62 of the body member 64 of the stent 60.

The stent 60 has two secondary arms 70 and 72 branching from the primary arm 62. Each secondary arm 70, 72 is formed by one of the coils 66, 68, respectively, the coils 66, 68 being separated from each other at a junction 74 to form the two secondary arms 70 and 72 of the stent 60.

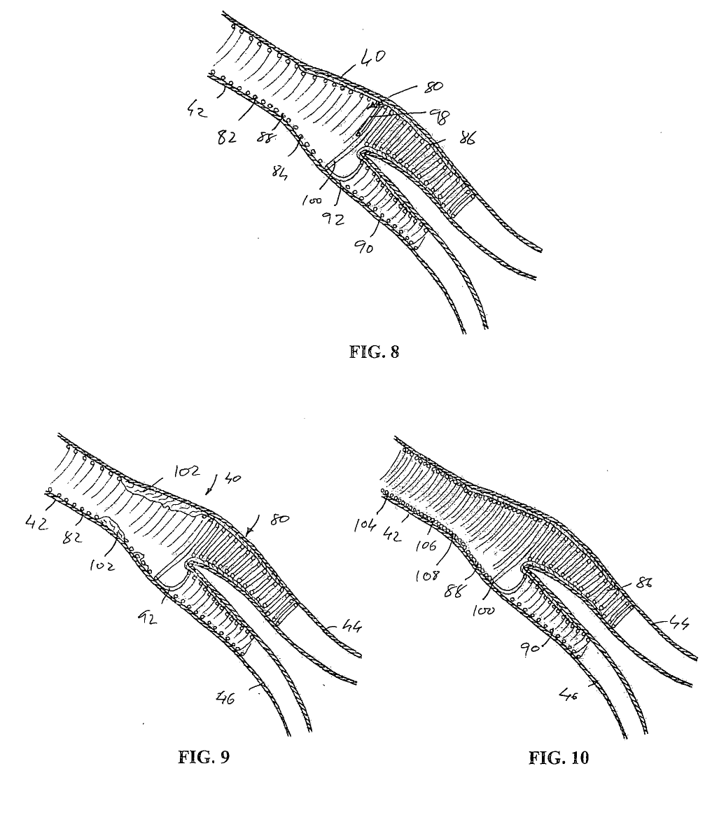

Referring now to FIGS. 5-10 of the drawings, yet a further embodiment of a support device used as a stent is described and is designated generally by the reference numeral 80 (FIG. 8). As in the previous embodiments, the stent 80 has a body member 82 made up of two substantially helically coiled elongate elements 84 and 86. The elongate element 84 is shown in FIG. 5 of the drawings in greater detail. The elongate element 84 has a first substantially helically coiled part 88 and a second substantially helically coiled part 90 distally arranged relative to the first coiled part 88. The parts 88 and 90 are interconnected by a bridging portion 92. The part 88 has a larger diameter than the part 90 so that a distal port 94 is formed at the distal end of the part 88. The part 88 may be made of a larger diameter than required. This allows the part 88 to fit into a tapered blood vessel as it need expand only as much as is required.

In use, the elongate element 84, in a non-deployed, unwound configuration, is placed in the lumen 24 of the delivery catheter 22 and is inserted into the blood vessel 40 to be delivered to the point of bifurcation of the blood vessel 40. Where the build up of plaque has occurred, the elongate element 84 is ejected from the distal end of the catheter 22 with the part 90 being received in the branch 46 of the blood vessel 40 where the part 90 coils to stent the branch 46 of the blood vessel 40 and to compress the plaque 48 in that branch 46. The part 88 of the elongate element 84 is then ejected from the delivery catheter 20 with the part 88 being received and coiled into its deployed configuration in the primary vessel 42 of the blood vessel 40.

As shown in FIG. 7 of the drawings, a further delivery catheter 96 is then used for delivering the elongate element 86 of the support device 80 into the branch 44 of the blood vessel 40. The delivery catheter 96 is fed through the port 94 of the part 88 into the branch 44 of the blood vessel 40. When a distal end of the delivery catheter 96 is located beyond the plaque 48, the catheter 96 is withdrawn proximally while the elongate element 86, which had been in an uncoiled, non-deployed configuration within the catheter 96, is ejected. As the catheter 96 is withdrawn, the elongate element 86 coils substantially helically as shown in FIG. 7 of the drawings until it adopts its fully coiled configuration as shown in FIG. 8 of the drawings.

A proximal turn 98 of the elongate element 86 meshes with a distal turn 100 of the part 88 to hold the elongate element 86 in position relative to the part 88 of the stent 80.

As shown in FIG. 9 of the drawings, restenosis can occur in the blood vessel 40. This is particularly so where the turns of the body member 82 of the stent 80 are spaced from each other. Thus, as shown, plaque 102 may build up through turns of the body member 82 of the stent, in due course. In such a situation, a further body member 104 (FIG. 10) is inserted into the primary arm 42 of the blood vessel 40 so that turns 106 of the body member 104 are interdigitated with turns 108 of the part 88 of the body member 82 of the stent 80 which compresses the plaque 102 in the blood vessel 40. It will be appreciated that similar body members (not shown) could be interdigitated with the parts 90 and 86 to deal with restenosis occurring in the branches 44 and 46, respectively, of the blood vessel 40.

Referring now to FIG. 13 of the drawings, reference numeral 110 designates a further embodiment of a support device. Once again, the support device 110 includes a body member 112 which can change from a non-deployed, unwound configuration to a deployed, substantially helically coiled configuration as shown in FIG. 13. The body member 112 is attached via a connector 114 to a tubular member 116. A distal end of the body member 112 is attached to a cable 118 which passes through the tubular member 116 and exits at a proximal end of the tubular member 116 externally of the patient's body. The cable 118 is reasonably rigid in an axial direction so that displacing the cable 118 axially with respect to the tubular member 116 in the direction of arrows 120 adjusts the spacing between adjacent turns 122 of the coiled body member 112, when in its deployed configuration. This may be particularly useful to access, for example, an intracranial aneurysm and to close over the neck of the aneurysm.

The cable 118 is also stiff in torsion so that, by rotating the cable 118 about its axis, the diameter of the body member 112 can be adjusted. Depending on the direction of rotation, the diameter of the body member 112 will either increase or decrease.

In certain circumstances, the body member 112 may be sequentially expanded and compressed in the blood vessel to compress plaque in the blood vessel.

For introduction of the support device 110 into the lumen of a delivery catheter (not shown), the tubular member 116 is withdrawn proximally with respect to the cable 118 to collapse the body 112 into its non-deployed configuration.

Those skilled in the art will readily appreciate that the blood vessels in which the support device is used are of small diameter. Typically, the diameter of these blood vessels can be as small as 3 mm and the thickness of wire 14 used to form the body member 12 of the support device 10 can be as little as 0.1 mm. It may be difficult to eject such a thin wire from the lumen 24 of the delivery catheter 22.

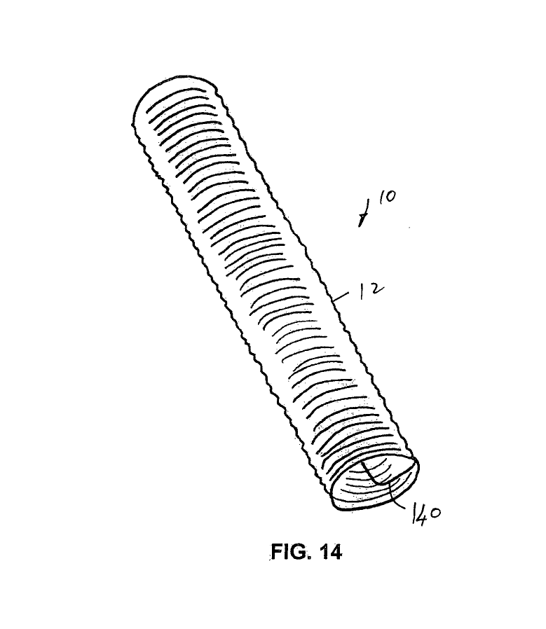

Thus, referring to FIGS. 14-16 of the drawings, yet a further embodiment of a system 20 for deploying a support device 10 is illustrated. With reference to FIGS. 1-2 of the drawings, like reference numerals refer to like parts, unless otherwise specified.

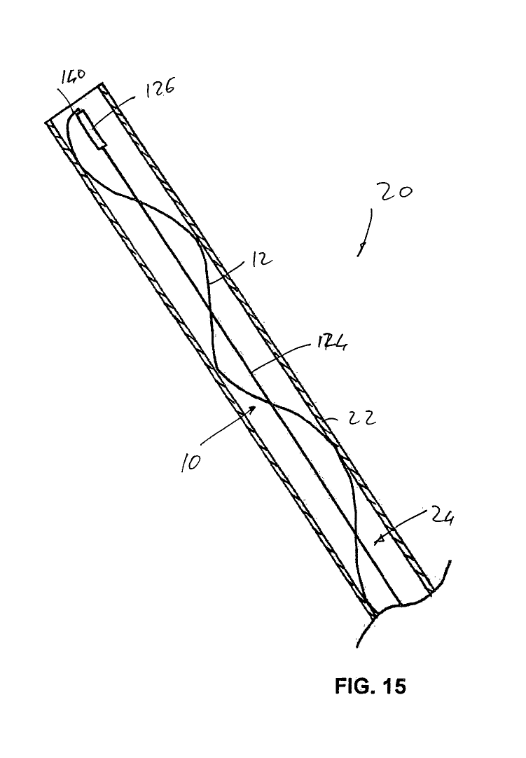

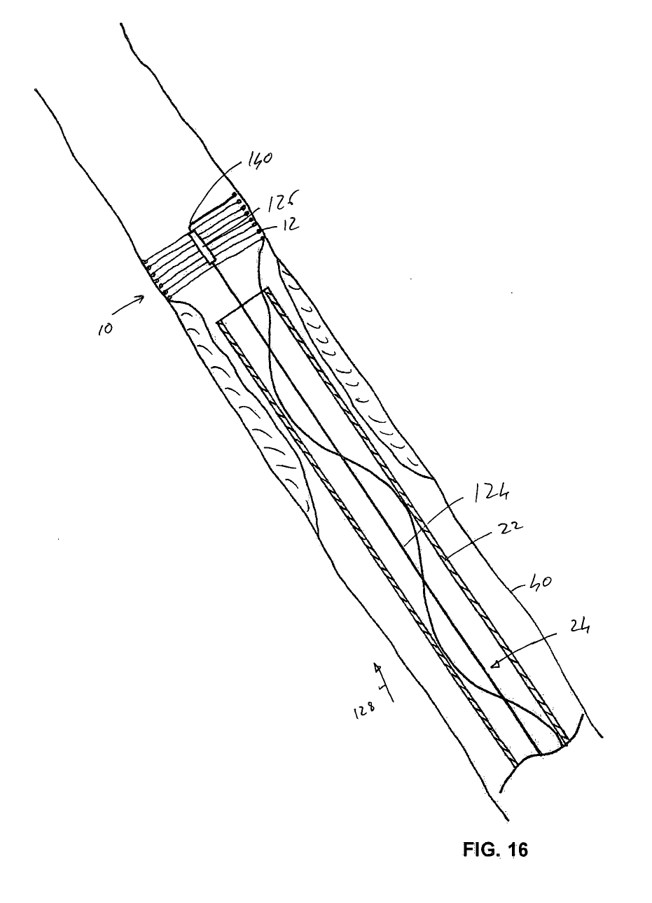

In this embodiment, a distal end of the body member 12 of the support device 10 includes an engageable formation 140. The engageable formation 140 is illustrated as a hook but could be any suitable shape or form to be engaged by an engaging member 126 carried at a distal end of a stylet 124 (FIGS. 15 and 16) receivable in the lumen 24 of the catheter 22.

The stylet 124 is received in the lumen 24 of the delivery catheter 22 of the system 20 together with the body member 12 of the support device 10 in its non-deployed configuration.

The engaging formation 126, which in this embodiment, is in the form of a tube 126, engages the hook 140.

While the catheter 22 is being maneuvered to the desired location in the vessel, the hook 140 is received in the tube 126 but both are contained within the catheter 22. At the site in the blood vessel 40, the stylet 124 is urged in the direction of arrow 128 out of the distal end of the catheter 22. This assists the body member 12 of the support device 10 in forming its coiled configuration as shown in FIG. 16 of the drawings as the body member 12 is ejected from the delivery catheter 22.

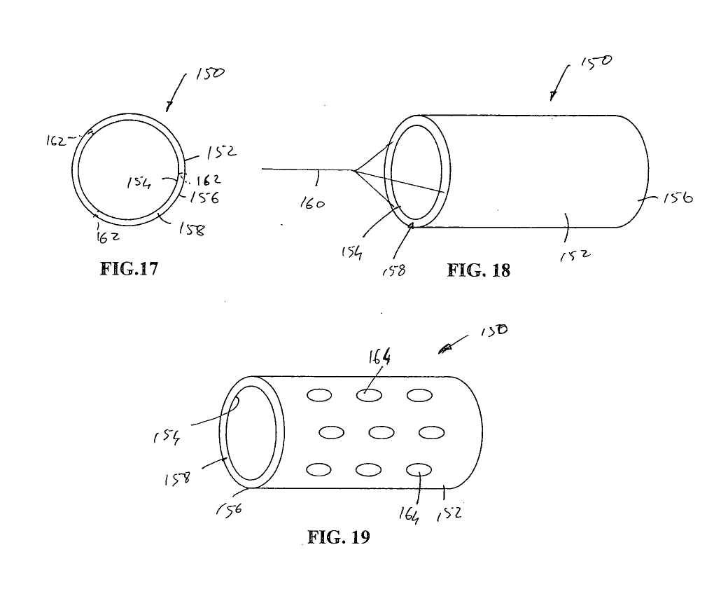

Referring now to FIGS. 17 and 18 of the drawings, a further embodiment of a support device 150 is illustrated. The support device 150 comprises a body member 152 having an inner wall defining member 154 and an outer wall defining member 156 arranged concentrically about the inner wall defining member 154. The wall defining members 154 and 156 are secured sealingly together along their abutting edges to form an annular, inflatable chamber 158. Thus, the support device 150 is an inflatable device which is placed in a tubular structure to be supported such as, for example, a blood vessel. A benefit of this embodiment is that, because the device is annular in transverse cross section, blood flow through the blood vessel is not occluded when the support device 150 is inflated.

The support device 150 is inflated, preferably, by means of a dye which is radiopaque to assist in detection of the support device 150 by a user of the support device 150.

The chamber 158 is inflated by inflating lines 160 which enter the chamber at various, spaced locations so that the lines 160 minimise impeding blood flow. Further, if desired, the annular chamber 158 may be divided into segments by dividing panels indicated in dotted lines at 162 so that segments of the chamber 158 can be inflated independently of one another.

The support device 150 can be used as a temporary stent in medical applications. When in its inflated condition, the support device 150 can be used to compress plaque on inner walls of the blood vessel 40.

Referring now to FIG. 19 of the drawings, yet a further embodiment of a support device is illustrated. With reference to FIGS. 17-18 of the drawings, like reference numerals refer to like parts, unless otherwise specified.

In this embodiment, the body member 152 of the support device defines a plurality of radially extending openings or pores 164. It will be appreciated that the periphery of each of these openings 164 is sealed so that inflating fluid cannot escape through the openings 164.

These openings 164 may be used where it is desired to insert items through the device 150, for example, to insert embolic coils in an aneurysm.

Yet a further advantage of the embodiment illustrated in FIG. 17-19 of the drawings is that the support device 150 can be easily collapsed within a delivery catheter without the need for excessive manipulation. Thus, when used as a temporary stent, withdrawal of the inflation fluid will cause the device 150 to collapse in on itself radially to enable it to be withdrawn into the delivery catheter without undue manipulation. Because the device 150 collapses radially inwardly when it is deflated, there is minimal likelihood of the device 150 damaging the inner wall of the blood vessel as it is withdrawn. Further, because of the largely uniform outer surface of the device 150, there is a more uniform stress distribution on the wall of the blood vessel. This is also of benefit in compacting vascular plaque in an evenly distributed manner.

If the device 150 is to be used to trap an embolic coil delivery catheter between the wall of the blood vessel and the device 150, the fact that the device is inflatable means that the device 150 can more readily conform to the shape of the delivery catheter thereby reducing the chance of damaging the wall of the blood vessel. Where the device 150 is used to embolise a aneurysm, the device 150 occludes the neck of the aneurysm allowing coagulation of blood in a sac of the aneurysm and stemming the flow of blood to the aneurysm.

In FIGS. 20a-20c an eleventh embodiment of a support device is illustrated and is designated generally by the reference numeral 170. With reference to the previous drawings, like reference numerals refer to like parts, unless otherwise specified.

In this embodiment, the support device 170 is intended for buttressing a blood vessel 172 having an aneurysm 174. The aneurysm 174 bulges from a side of the blood vessel 172 and has a neck 176.

To embolise the aneurysm 174, embolic coils 178 are inserted into a sac of the aneurysm 174 through the neck 176 using a delivery catheter (not shown). To support the embolic coils 178 in the sac of the aneurysm 174, the support device 170 is inserted into the blood vessel 172 using the delivery catheter 22. Sufficient turns 14 of the body member 12 of the support device 170 are ejected from a distal end of the catheter 22 in the region of the neck 176 of the aneurysm 174 to retain the embolic coils 178 in the sac of the aneurysm 174 and to inhibit the embolic coils 178 from protruding into the blood vessel 172.

The number of coils 14 of the body member 12 which are ejected from the delivery catheter 22 depend on the size of the neck 176 of the aneurysm. Thus, as shown in FIGS. 20a-20c, various numbers of coils 14 of the body member 12 of the support device 170 are ejected from the catheter 22 depending on the size of the neck 176.

In use, once the embolic coils 178 have been inserted into the sac of the aneurysm 174, blood in the sac of the aneurysm 174 thromboses and, after stasis, the support device 170 is retracted into the catheter 22 by uncoiling the body member 12 by pulling proximally on the retrieval wire 28. If necessary, the delivery catheter 22 is displaced distally to assist in withdrawing the body member 12 into the lumen 24 of the catheter 22 for removal from the blood vessel 172. Advantageously, because the body member 12 collapses radially as it is converted to its non-deployed configuration, the likelihood of damaging the internal wall of the blood vessel 172 by scraping or dragging is reduced.

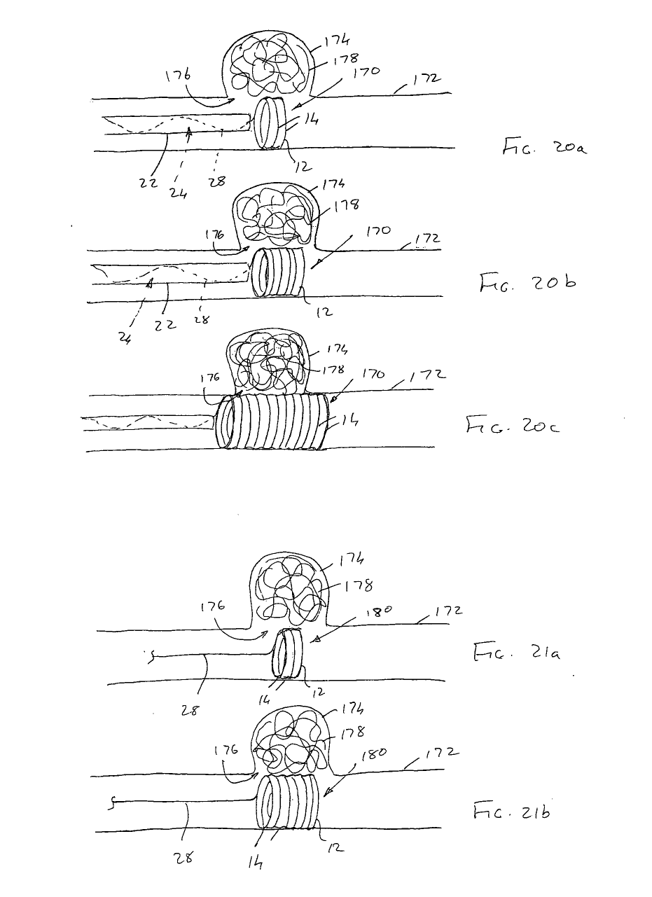

Referring to FIGS. 21a-21b of the drawings, two versions of a twelfth embodiment of a support device 180 are shown. With reference to FIGS. 20a-20c, like reference numerals refer to like parts, unless otherwise specified.

In this embodiment, instead of only a portion of the support device being ejected from the delivery catheter as required to buttress the blood vessel in the region of the aneurysm 174, the support device 180 is selected having a body member 12 of a size suitable to at least partially occlude the neck 176 of the aneurysm 174 to retain the embolic coils 178 within the sac of the aneurysm and to inhibit the embolic coils from protruding into the blood vessel 172.

Generally, the body member 12 need not fully occlude the neck 176 of the aneurysm 174 but need only support the embolic coils 178 out of the path of the blood vessel 172. As shown in FIG. 21a of the drawings, a body member 12 of fewer turns 14 is provided than in the case of the embodiment shown in FIG. 21b of the drawings.

As in the case of the previous embodiment, this embodiment also makes use of the delivery catheter 22 although it is not shown. The retrieval wire 28 protrudes through the lumen 24 of the delivery catheter 22 to enable the support device 180 to be removed after stasis has occurred in the sac of the aneurysm 174.

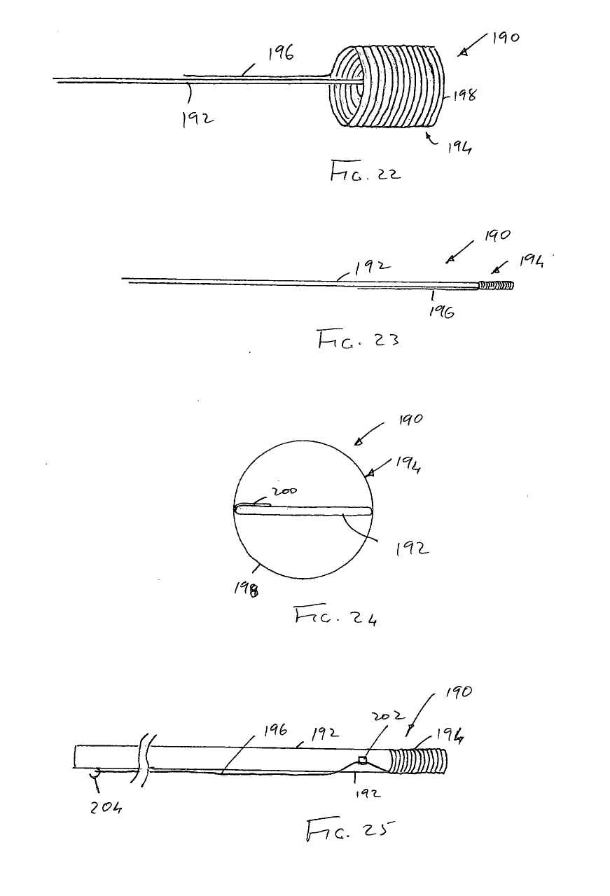

FIGS. 22-25 show a further embodiment of a system for deploying a support device and is designated generally by the reference numeral 190. The system 190, instead of using a delivery catheter, uses an introducer in the form of a delivery wire or rod 192. The delivery rod 192 can have any suitable cross-sectional shape when viewed end on but, as illustrated in FIG. 24 of the drawings, in this example has a ribbon-like or strip-like cross-section.

The system 190 includes a substantially helically coiled support device 194. In its non-deployed configuration, the coiled support device 194 is tightly wound about a distal end of the delivery rod 192 to be constricted both radially and axially in comparison with the deployed configuration of the support device 194. The support device 194 also includes an integrally formed, proximally extending manipulating formation in the form of a retrieval wire 196 which extends along the delivery rod 192 to project out of the patient's body and which enables a clinician to manipulate the support device 194.

A distal turn 198 of the support device 194 has a tail 200 (FIG. 24) which is fast with the delivery rod 192.

Further, the system 190 includes a retaining member in the form of a notch or elevated portion 202 on the delivery rod 192 through which the retrieval wire 196 projects to assist in retaining the support device 194 in its non-deployed, tightly coiled configuration.

In use, the delivery rod 192 is inserted through the vasculature of the patient's body to deliver the support device, which is in its tightly coiled, non-deployed configuration on the delivery rod 192, to the site in the patient's body where a blood vessel is to be supported.

Once the support device 194 has been delivered to the site in the blood vessel, a clinician operates a release mechanism, such as a latch 204, to unlatch the retrieval wire 196 from the delivery rod 192 and, while retaining control of the retrieval wire 196 allows the support device 194 to expand radially and axially to buttress the blood vessel at the desired location. The clinician is able to adjust the spacing between the coils of the support device 194 by relative manipulation between the delivery rod 192 and the retrieval wire 196.

Upon completion of the insertion of the embolic coils into the aneurysm and after stasis has occurred, the clinician pulls on the retrieval wire 196 to collapse the support device 194 to a retrieval configuration. In the retrieval of the support device 194, it may not be that the support device 194 adopts the constricted, coiled configuration but may merely be retrieved by collapsing the support device into a drawn out semi-straightened configuration and withdrawing the support device together with the delivery rod 192.

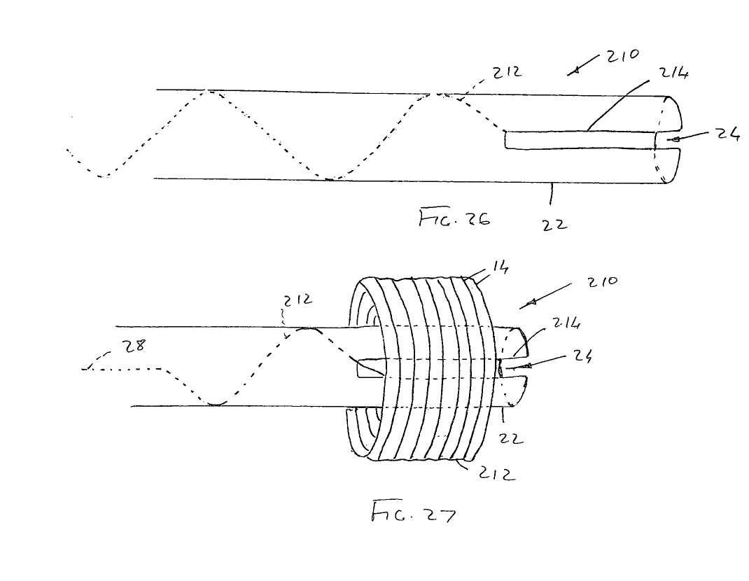

Referring now to FIGS. 26 and 27 of the drawings, a fourth embodiment of a system for deploying a support device is illustrated and is designated generally by the reference numeral 210. In this embodiment, the system 210 includes the delivery catheter 22 defining a lumen 24 in which a support device 212 is contained, in a non-deployed, semi-straightened configuration as illustrated in FIG. 26 of the drawings.

In this embodiment, the delivery catheter 22 includes a guide formation in the form of an axially extending slot 214 at the distal end of the delivery catheter 22. When the support device 212 is ejected from the distal end of the catheter, it is ejected though the slot 214 which encourages each coil of the support device 212 to form in a plane substantially normal to a longitudinal axis of the delivery catheter 22. The guide slot 214 therefore assists in orientating the coils 14 in the desired position in the blood vessel (not shown).

Once again, in this embodiment, the support device 212 includes the manipulating formation in the form of a retrieval wire 28 to enable the support device 212 to be retracted into the lumen 24 of the delivery catheter 22 upon completion of the procedure.

In FIGS. 28-30 of the drawings, a fifth embodiment of a system for deploying a support device is illustrated and is designated generally by the reference numeral 220.

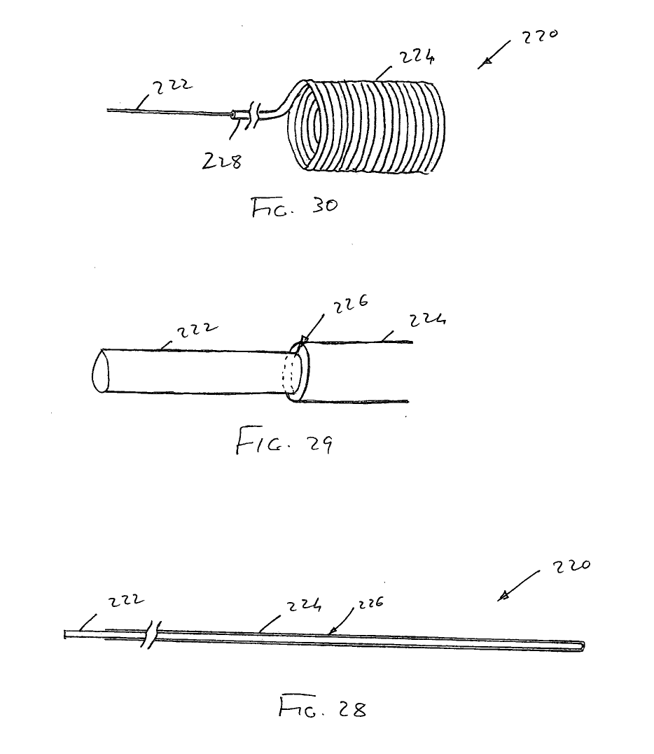

In this embodiment, the system 220 includes an introducer in the form of a delivery rod 222. A support device 224 to be delivered to the site in the blood vessel of the patient is, unlike the previous embodiments, a tubular support device 224 defining a passage 226 in which the delivery rod 222 is receivable as shown more clearly in FIG. 29 of the drawings.

The support device 224 is heat set into a coiled configuration as shown in FIG. 30 of the drawings. However, for delivery of the support device 224 to the site, the delivery rod 222 is inserted into the passage 226 of the support device 224 causing it to straighten out to adopt the non-deployed configuration shown in FIG. 28 of the drawings. It will be appreciated that, in this embodiment, a separate delivery catheter is not required and the system, as shown in FIG. 28 of the drawings can be steered through the vasculature of the patient to deliver the support device 224 to the desired site. The delivery rod 222 therefore acts in the same manner as a guide wire.

When the distal end of the system 220 is at the desired site, the delivery rod 222 is retracted proximally relative to the support device 224. To enable this to occur, the support device 224 has a proximally extending manipulating member 228 which protrudes out of the patient's body so that the clinician can access the manipulating member 228 to effect relative displacement between the support device 224 and the delivery rod 222.

As the delivery rod 222 is retracted from the support device 224, the support device 224 coils to adopt its deployed configuration as shown in FIG. 30 of the drawings to buttress the blood vessel in which the support device 224 is contained.

After completion of the procedure, the delivery rod 222 is displaced distally with respect to the support device 224 to uncoil the support device 224 to enable it to be retracted from the patient's body. Once again, due to this procedure, the likelihood of the support device 224 scraping or dragging along the wall of the blood vessel is reduced thereby minimising the risk of damage to the blood vessel during withdrawal of the support device 224.

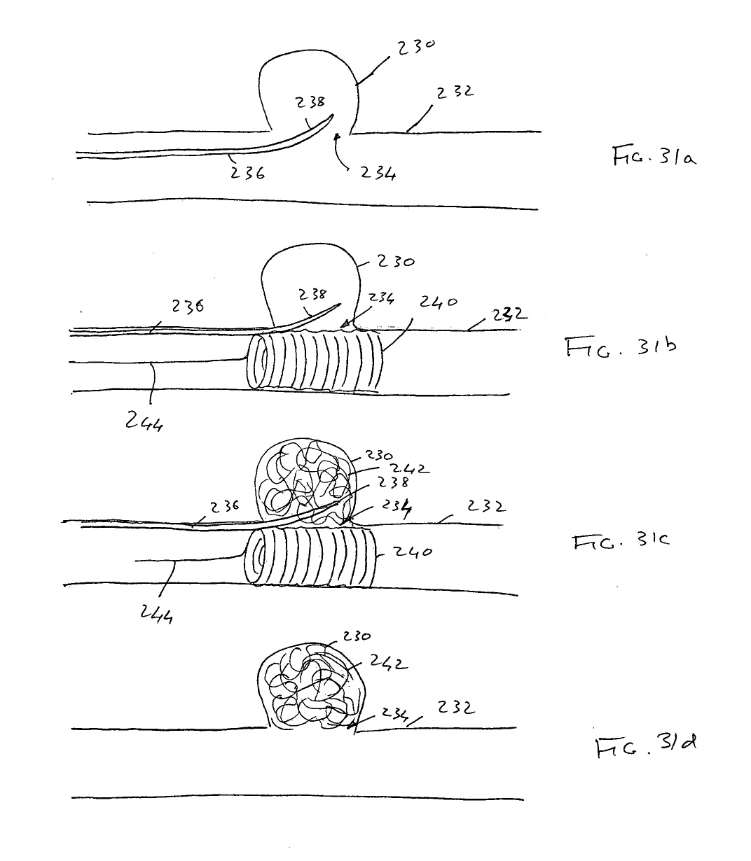

Referring finally to FIGS. 31a-31d of the drawings, an embodiment of a system for, and a method of, embolising an aneurysm is illustrated.

In this embodiment, an aneurysm 230 is shown in a blood vessel 232. The aneurysm has a neck 234 opening out into a passage of the blood vessel 232.

As an initial step in the method, an introduction catheter is introduced into the patient's vasculature and is steered through the vasculature so that the catheter 236 is received in the blood vessel 232. A distal end 238 of the catheter 236 is inserted into a sac of the aneurysm 230.

A support device 240 is introduced into the vasculature of the patient using an introducer (not shown) with the support device 240 being in a non-deployed, collapsed configuration to facilitate its insertion into the blood vessel 232. At the site of the sac of the aneurysm 230, the introducer is removed so that the support device 240 adopts its coiled, deployed configuration as illustrated in FIG. 31b of the drawings. The support device 240 is selected to have a transverse dimension to trap the distal end 238 of the introduction catheter 236 in position relative to the aneurysm 230.

The support device 240 serves the additional purpose of being positioned across the neck 234 of the aneurysm 230 and may extend over the full opening of the neck 234 or part thereof. It is sufficient that the support device 240 is positioned relative to the neck 234 of the aneurysm 230 to retain subsequently delivered embolic coils 242.

Thus, once the support device 240 has been deployed, embolic coils 242 are ejected from the distal end 238 of the introduction catheter 236. The introduction of the embolic coils 242 allows thrombus to occur in the sac of the aneurysm 230. Once stasis has occurred, the support device 240 is withdrawn by pulling on a manipulating member in the form of a retrieval wire 244. Pulling on the retrieval wire 244 which, in use, extends through the delivery catheter results in the support device 240 collapsing into its non-deployed configuration facilitating withdrawal of the support device 240 into the delivery catheter and removal from the blood vessel 232. After withdrawal of the support device 240 and, because stasis has occurred, the embolic coils 242 are retained within the sac of the aneurysm 230.

In due course, endothelial cells originating from the parent blood vessel 232 migrate over the surface of the thrombus in the sac of the aneurysm 230 thereby covering the aneurysm neck 234. The support device 240 thus, advantageously, retains the embolic coils 242 within the sac of the aneurysm 230 allowing regrowth of the endothelial cells which may, otherwise be inhibited by parts of the embolic coils protruding through the neck 234 of the aneurysm.

It will be appreciated that, in all the coiled embodiments described above, the ratios wire major transverse dimension/thickness to coil major transverse dimension described with reference to the first embodiment apply.

It is an advantage of the various embodiments of the support device described above that a versatile support device is provided which is easily positioned in a blood vessel to be supported or buttressed. The structure of the support device illustrated in FIGS. 1, 4-13 and 14 means that the device is versatile and easy to position. Shorter or longer devices can be made as desired and the devices can be made with turns of the coil abutting or being spaced from each other as required and can be selected or manipulated to different major transverse dimensions as required.

Such a construction also facilitates the placement of items through the support device. Thus, items such as embolic coils can be inserted through the support device and, for example, into an aneurysm in the wall of the blood vessel for coagulating blood in the sac of the aneurysm. Still further, instruments can be inserted between adjacent turns of the coil to access the aneurysm if desired.

The construction of these embodiments is also advantageous in that the support device can be readily collapsed to be withdrawn from the location within the blood vessel once the support device is no longer needed. This is beneficial in that a foreign body is not left within the blood vessel and the passage of blood through the blood vessel is not impeded by the continued presence of the support device.

This latter advantage applies also to the embodiment of the support device shown in FIG. 17-19 of the drawings.

In the case of the coiled support devices, each of these device has the further advantage of being flexible to be able to follow the curvature of a blood vessel. Thus the need for multiple, shorter support devices is obviated. Standard stents (non flexible) when placed in a curved portion of a vessel will substantially straighten the portion of the vessel putting undue stress on the walls of the blood vessels which is particularly disadvantageous where the wall has been weakened, for example, due to an aneurysm. Because, the coiled support device can conform to the curvature of the blood vessel this reduces stress on the wall of the blood vessel and minimises changes to the fluid dynamics through the blood vessel.

Yet a further advantage of the coiled configuration of the support device is that, when it is withdrawn into the delivery catheter for removal, relative movement occurs by urging the delivery catheter in a distal direction while withdrawing the support device proximally into the interior of the lumen of the delivery catheter. The likelihood of the support device being dragged along the inner surface of the wall of the tubular structure is minimised thereby minimising the risk of damaging the tubular structure.

Another major advantage is that, when the coiled support device is uncoiled or unwound, it has a very small transverse dimension. Thus a narrower delivery catheter can be used than has previously been used. This minimises the risk of damage to a patient's vasculature when the delivery catheter is maneuvered through the vasculature.

It will be appreciated by persons skilled in the art that numerous variations and/or modifications may be made to the described specific embodiments without departing from the scope of the appended claims. The present embodiments are, therefore, to be considered in all respects as illustrative and not restrictive.

1. A blood vessel support device which includes a body member, the body member comprising at least one elongate element of a settable material, the at least one elongate element being configured to change from a non-deployed configuration to be carried by an introducer for delivery to a site within a blood vessel to a deployed, coiled, tubular configuration at the site within the blood vessel.

2. The device of claim 1 in which the, or each, elongate element is in the form of a wire capable of adopting a coiled configuration.

3. The device of claim 2 in which the, or each, wire is of at least one of a settable metal material and a settable plastics material.

4. The device of claim 1 in which the, or each, elongate element is hollow to define a passage in which the introducer is received to retain the elongate element in its non-deployed configuration.

5. The device of claim 1 in which the at least one elongate element is shaped so that, when the at least one elongate element is in its deployed configuration, at least one opening is formed between adjacent turns of a coil formed by the deployment of the body member, the at least one opening enabling access to be gained to the site through the body member.

6. The device of claim 1 in which the body member comprises a plurality of interdigitated elongate elements.

7. The device of claim 1 in which the body member forms at least two parts when in its deployed configuration, a primary part and at least one secondary part, the parts being axially spaced and interconnected by a bridging portion.

8. The device of claim 7 in which a diameter of the primary part differs from a diameter of the at least one secondary part.

9. The device of claim 7 which includes two secondary parts extending from a distal end of the primary part to form a bifurcated body member.

10. The device of claim 1 in which the body member includes an adjustment mechanism attached to the body member for adjusting a diameter of the body member when the body member is in its deployed configuration.

11. The device of claim 1 in which the body member includes a manipulating formation for enabling the body member to be manipulated and retrieved from the site.

12. The device of claim 11 in which the manipulating formation is an elongate member extending proximally from the body member, a proximal end of the elongate member being accessible externally of a patient's body.

13. The device of claim 1 in which the body member includes an engageable formation to be engaged by an engaging member to assist in arranging the body member at least in its deployed configuration.

14. A system for deploying a support device in a passage of a blood vessel, the system including
an introducer; and
a support device, as claimed in any one of the preceding claims, carried by the introducer with the body member in its non-deployed configuration.

15. The system of claim 14 which includes an engaging member associated with the introducer for engaging the support device to assist in removing the support device from the introducer to enable the support device to adopt its deployed configuration after ejection from the introducer.

16. The system of claim 14 in which the introducer is in the form of a delivery catheter, withdrawal of the catheter from a site after delivery of the support device to the site in the passage of the vessel assisting in causing the body member to adopt its deployed configuration.

17. The system of claim 14 in which the introducer is a wire about which the support device, in its non-deployed configuration, is mounted in a tightly wound configuration, the support device being released from its non-deployed configuration to expand at least radially into its deployed configuration.

18. The system of claim 14 in which the introducer is a wire and the support device is tubular.

19. The system of claim 18 in which the support device, in its non-deployed configuration, is received over the introducer and is released from a distal end of the introducer by relative movement between the introducer and the support device to enable the support device to adopt its deployed configuration.

20. The system of claim 12 in which the support device is a primary stent and in which the support device includes at least one secondary stent having a body member of a similar configuration to the primary stent, the primary stent and the secondary stent being used together to form a bifurcated stent at the site.

21. A method of deploying a support device in a passage of a blood vessel, the method including
delivering the support device, as claimed in claim 1, to a site in the vessel using an introducer, the body member of the support device being in its non-deployed configuration; and
ejecting the support device from a distal end of the introducer to enable the support device to adopt its deployed configuration.

22. The method of claim 21 which includes withdrawing the introducer from the site in the vessel at the same time as ejecting the support device from the introducer to aid in the body member of the support device adopting its deployed configuration.

23. The method of claim 21 which includes ejecting only a portion of the support device from the introducer so that that portion of the support device adopts its deployed configuration

24. The method of claim 21 which includes selecting a size of support device suitable for the size of the region of the blood vessel to be supported.

25. The method of claim 21 which includes engaging an end of the body member of the support device to assist in ejecting the support device from the introducer.

26. The method of claim 21 which includes accessing a manipulating formation of the support device protruding from the site, causing the device to adopt its non-deployed configuration and withdrawing the device from a patient's body.

27. The method of claim 21 which includes ejecting a first support device from the introducer at the site and, thereafter, ejecting a second support device from the introducer so that the second support device adopts a desired position relative to the first support device at the site.

28. The method of claim 27 which includes connecting at least a portion of the second support device to the first support device to retain the support devices in position relative to each other.

29. The method of claim 27 which includes arranging the second support device relative to the first support device to form a bifurcated support device.

30. The method of claim 29 which includes connecting the second support device to the first support device by interdigitating at least a portion of the second support device with a portion of the first support device.

31. A blood vessel support device which includes an inflatable body member, the body member being annular when viewed end-on.

32. The support device of claim 31 in which a wall of the body member defines at least one opening, sealed about its periphery to inhibit escape of inflating fluid through the opening.

33. The support device of claim 31 in which the body member comprises a plurality of panels, at least some of which are inflatable independently of one another.

34. A system for embolising an aneurysm, the system including
an introducer;
a support device, as described above, carried by the introducer with the body member of the support device being carried by the introducer in its non-deployed configuration; and
an introduction catheter, in which embolising equipment is receivable, the support device being dimensioned to hold a distal end of the introduction catheter captive between a wall of the blood vessel and the support device when the support device is in its deployed configuration.

35. A method of embolising an aneurysm, the method including
delivering a support device, as described above, to a site in the vessel using an introducer, the body member of the support device being carried in its non-deployed configuration by the introducer during the delivery process;
introducing an introduction catheter containing embolising equipment to the site; and
ejecting the support device from a distal end of the introducer to enable the support device to adopt its deployed configuration and to hold a distal end of the introduction catheter captive between a wall of the blood vessel and the support device in its deployed configuration.
